(19) United States
(12) Patent Application Publication
(10) Pub. No.: US 2026/0007445 A1
(43) Pub. Date: Jan. 8, 2026

(54) IMPACTOR DESIGN FOR BIDIRECTIONAL IMPACT FORCE

(71) Applicants: Smith & Nephew, Inc., Memphis, TN (US); Smith & Nephew Orthopaedics AG, Zug (CH)

(72) Inventors: Eric Ashuckian, Pittsburgh, PA (US); Giorgos Karatziolas, London (GB); Ruben Doyle, London (GB)

(73) Assignees: Smith & Nephew, Inc., Memphis, TN (US); Smith & Nephew Orthopaedics AG, Zug (CH)

(21) Appl. No.: 19/243,900

(22) Filed: Jun. 20, 2025

Related U.S. Application Data
(60) Provisional application No. 63/666,762, filed on Jul. 2, 2024.

(51) Int. Cl. A61B 17/92

(52) U.S. Cl. CPC A61B 17/92; A61B 2017/924

(57) ABSTRACT

An impactor and methods of operation of an impactor. The impactor includes an electromagnetic component having a stationary electromagnetic housing and a moving armature component. The housing includes a coil that receives an electric current resulting in generation of an electromagnetic field for triggering translation of the armature within the housing. The impactor includes a striker body coupled to the armature. The striker body includes a distal end. The impactor includes a strike chamber having an interior and a distal strike wall disposed at a distal end of the chamber, and a spacer disposed at a proximal end of the chamber and opposite the distal strike wall. The electromagnetic field forces the armature and the striker body to translate in at least one direction. Translation of the armature causes the distal end of the striker body to strike at least one of the distal strike wall and the spacer.

### CROSS-REFERENCE TO RELATED APPLICATIONS

This is a non-provisional of, and claims the benefit of the filing date of, pending U.S. Provisional Patent Application No. 63/666,762, filed Jul. 2, 2024, entitled “Impactor Design for Bidirectional Impact Force,” the entirety of which application is incorporated by reference herein.

### TECHNICAL FIELD

The present disclosure is generally directed to a surgical impactor, and, in particular, to a surgical impactor having an electromagnetic component for generating of an impact energy.

### BACKGROUND

Orthopedic surgical procedures such as, for example, hip procedures, knee procedures, shoulder procedures, etc., have become common place in today's society. For example, total hip arthroplasty or hip replacement is a well-known procedure for repairing damaged bone (e.g., a damaged hip). During a total hip arthroplasty, an acetabular system may be implanted into a patient's acetabulum. In addition, and/or alternatively, a femoral implant may be implanted into a patient's femur. During the surgical procedure, the patient's bone typically needs to be prepared to receive the orthopedic implant. For example, a surgical tool such as, for example, an orthopedic broach, rasp, cutting tool, etc. (terms used interchangeably herein without the intent to limit or distinguish) may be used to prepare an inner surface of a patient's intramedullary canal to receive an orthopedic implant such as, for example, a femoral hip prosthesis, an intramedullary nail, etc. The preparation of the intramedullary canal by the surgeon is designed to insure a proper fit between the patient's femur and the implant. In addition, the orthopedic implant such as, for example, the acetabular cup, may need to be impacted into proper position. Moreover, during removal of the broach from the patient's intramedullary canal, the broach may become struck within the patient's intramedullary canal.

A surgical impactor may be used for insertion and/or removal of implants. Some existing impactors rely on motors to deliver energy for such insertion/removal. Other impactors use solenoid-based systems that produce electromagnetic energy that is applied to deliver the necessary force to objects coupled to the impactor (e.g., tools, implants, etc.), and hence, perform requisite surgical procedures. The solenoid-based impactor systems suffer from reverse direction movements that may negate effects (e.g., positioning of an implant) that may be intended by the user of the impactor systems. As such, it would be beneficial to provide a surgical impactor tool that reduces such reverse direction movements.

### SUMMARY

This summary is provided to introduce a selection of concepts in a simplified form that are further described below in the detailed description. This summary is not intended to identify key features or essential features of the claimed subject matter, nor is it intended as an aid in determining the scope of the claimed subject matter.

In some examples, the present disclosure relates to an impactor. The impactor may include an electromagnetic component including a stationary electromagnetic housing and a moving armature component. The stationary electromagnetic housing may include a coil configured to receive an electric current resulting in generation of an electromagnetic field for triggering translation of the armature component within the stationary electromagnetic housing. The impactor may also include a striker body configured to be coupled to the armature component and an object. The striker body may include a distal end. The impactor may further include a strike chamber having an interior and a distal strike wall disposed at a distal end of the strike chamber, and a spacer disposed at a proximal end of the strike chamber and opposite the distal strike wall. The electromagnetic field may be configured to force the armature component and the striker body to translate in at least one direction. Translation of the armature component may be configured to cause the distal end of the striker body to strike at least one of the distal strike wall and the spacer.

In any preceding or subsequent examples, at least one direction of translation movement of the armature component and the striker body may be directly and/or indirectly dependent on a direction of the electric current applied to the coil.

In any preceding or subsequent examples, a change in a direction of the electric current applied to the coil may be configured to change at least one direction of translation movement of the armature component and the striker component.

In any preceding or subsequent examples, at least one direction may include a forward impact direction, a reverse impact direction, a combination of forward and reverse impact directions, or any combination thereof.

In any preceding or subsequent examples, the object may include at least one of: a tool, an implant, or any combination thereof.

In any preceding or subsequent examples, translation movement of the striker body may be configured for positioning an implant in a bone and/or removal of the implant from the bone.

In any preceding or subsequent examples, the coil may be configured to receive one or more current pulses to trigger translation of the armature component and the striker body.

In any preceding or subsequent examples, one or more current pulses may include a high current pulse and a low current pulse, wherein the low current pulse is smaller than the high current pulse. A duration of application of the high current pulse may be longer than a duration of application of the low current pulse. The low current pulse may be configured to prevent movement of the armature component and the striker body in a direction opposite to a direction of movement of the armature component and the striker body resulting from application of the high current pulse.

In any preceding or subsequent examples, the high current pulse and the low current pulse may be applied intermittently.

In any preceding or subsequent examples, the spacer may be configured to limit a translation distance of the distal end of the striker body within the interior of the strike chamber.

In any preceding or subsequent examples, the impactor may include one or more sensors that may be configured to detect position of the armature component within the stationary electromagnetic housing. The direction(s) of translation of the armature component may be determined based on the position detected by the one or more sensors.

In some examples, the present disclosure relates to an impactor. The impactor may include an electromagnetic component including a stationary electromagnetic housing and a moving armature component. The stationary electromagnetic housing may include a coil configured to receive an electric current resulting in generation of an electromagnetic field for triggering translation of the armature component within the stationary electromagnetic housing. The impactor may also include a striker body configured to be coupled to the armature component and an object, the striker body including a distal end, and a strike chamber having an interior and a distal strike wall disposed at a distal end of the strike chamber. The electromagnetic field may be configured to force the armature component and the striker body to translate in at least one direction. Translation of the armature component may be configured to cause the distal end of the striker body to strike the distal strike wall. The impactor may include one or more sensors configured to determine position of the moving armature component, and at least one processor configured to determine an amount and a direction of the electric current to apply to the coil to generate the electromagnetic field based on data associated with the determined position of the moving armature component and received from the one or more sensors.

In any preceding or subsequent examples, the impactor may include the direction may include at least one of: a forward impact direction, a reverse impact direction, a combination of forward and reverse impact directions, or any combination thereof.

In any preceding or subsequent examples, the amount and the direction of current may be determined at at least one of: prior to the translation of the armature component for delivery of an impact by the impactor, during the translation of the armature component for delivery of the impact, during the translation of the armature component after delivery of the impact, or after the translation of the armature component.

In any preceding or subsequent examples, the impactor may include one or more sensors may include at least one of: a position sensor, a pressure sensor, an infrared sensors or combination thereof.

In any preceding or subsequent examples, in response to at least one processor determining the amount and the direction of the electric current, the coil may be configured to receive one or more current pulses to trigger translation of the armature component and the striker body. One or more current pulses may include a high current pulse and a low current pulse. The low current pulse may be smaller than the high current pulse.

In any preceding or subsequent examples, a duration of application of the high current pulse may be longer than a duration of application of the low current pulse. Application of the low current pulse may be configured to prevent movement of the armature component and the striker body in a direction opposite to a direction of movement of the armature component and the striker body resulting from application of the high current pulse.

In any preceding or subsequent examples, the high current pulse and the low current pulse may be applied intermittently.

Examples of the present disclosure provide numerous advantages. For example, the current subject matter's use of the spacer arranged and configured within the striker chamber for interaction with the distal end of the striker body may be configured to control translational movement of the striker body and hence, the distal connector that may be coupled to one or more objects, tools, implants, etc. Such control may reduce an unwanted movement of the distal connector subsequent to initial impact, which may affect the intent and result of the surgical procedure. For example, the current subject matter may be configured to limit or prevent reverse direction translation of the distal connector and hence, for example, an implant coupled to the distal connector, during positioning of the implant in a bone of the patient (and vice versa).

Further features and advantages of at least some of the examples of the current subject matter, as well as the structure and operation of various examples of the current subject matter, are described in detail below with reference to the accompanying drawings.

### BRIEF DESCRIPTION OF THE DRAWINGS

The accompanying drawings, which are incorporated in and constitute a part of this specification, show certain features of the subject matter disclosed herein and, together with the description, help explain some of the principles associated with the disclosed implementations. In the drawings,

FIG. 1 illustrates an exemplary impactor that may be configured to incorporate a system for reducing electromagnetic eddy currents during operation of the impactor, in accordance with one or more features of the present disclosure;

FIGS. 2a-e illustrate further details of the impactor shown in FIG. 1, in accordance with one or more features of the present disclosure;

FIGS. 3a and 3b illustrate various views of a spacer of the impactor shown in FIG. 1, in accordance with one or more features of the present disclosure;

FIG. 4 is a block diagram of an exemplary orthopedic surgical instrument and/or impactor in accordance with one or more features of the present disclosure;

FIG. 5 illustrates an exemplary computing apparatus in accordance with one or more features of the present disclosure;

FIG. 6 illustrates an example of a storage medium to store impactor logic in accordance with one or more features of the present disclosure; and

FIG. 7 illustrates an example computing platform in accordance with one or more features of the present disclosure.

It should be understood that the drawings are not necessarily to scale and that the disclosed examples are sometimes illustrated diagrammatically and/or in partial views. In certain instances, details that are not necessary for an understanding of the disclosed methods and devices or which render other details difficult to perceive may have been omitted. It should be further understood that this disclosure is not limited to the particular examples illustrated herein. In the drawings, like numbers refer to like elements throughout unless otherwise noted.

### DETAILED DESCRIPTION

To address these and potentially other deficiencies of currently available solutions, one or more implementations of the current subject matter relate to methods, systems, articles of manufacture, and the like that can, among other possible advantages, provide an orthopedic surgical instrument or impactor, and in particular, a surgical impactor tool that may be configured to detect whether a forward or a reverse impact is desired and thus, be able to operate in a bidirectional mode.

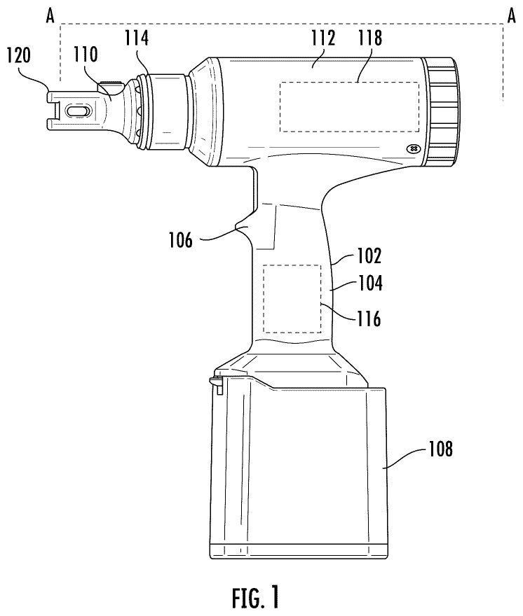

It should be appreciated that while, for example, detection of whether a forward or a reverse impact is desired in accordance with the present disclosure may be described herein in connection with an orthopedic surgical impactor (e.g., as shown in FIG. 1) that may be used to, for instance, drive a broach into a patient's bone to, for example, prepare an intramedullary canal of the patient's bone, the present disclosure is not so limited and such detection and subsequent bidirectional operation (as shown and described hereinbelow) may be used in connection with any tool, device, instrument, etc. now known or hereafter developed such as, for example, a tap used to insert a bone screw, construction hand tools or power tools, etc. As such, the present disclosure should not be limited to any particular tool unless explicitly claimed.

The following description of an orthopedic impactor (e.g., as shown in and discussed in connection with FIG. 1) is provided here for exemplary, illustrative purposes only and is not intended to limit the current subject matter and/or any of its elements, applications and/or advantages.

With reference to FIG. 1, an example of an orthopedic surgical instrument, impactor, or impactor mechanism (terms used interchangeably herein without the intent to limit or distinguish) is disclosed. In use, the orthopedic impactor is arranged and configured to transmit a forward energy or motion to, for example, drive a surgical tool (e.g., a broach) or implant into a patient's bone, and a reverse energy or motion to, for example, remove a struck or lodged surgical tool (e.g., a broach) or implant from a patient's bone.

The orthopedic impactor may be arranged and/or configured to position, insert and/or implant an orthopedic implant such as, for example, but not limited to, an acetabular cup, an intramedullary nail, a femoral hip implant, etc. into a bone matter (e.g., a bone of a patient). Alternatively, or in addition to, the orthopedic impactor may be coupled to a surgical tool such as, for example, but not limited to, a broach, to prepare a bone to receive an orthopedic implant.

The orthopedic impactor may cause application of a forward energy and/or motion to drive an orthopedic implant and/or surgical tool into a patient's bone. In addition, the orthopedic impactor may be arranged and/or configured to cause application of a reverse energy and/or motion to, for example, but not limited to, remove a stuck and/or lodged surgical tool and/or implant from a patient's bone. The orthopedic impactor may be configured to allow selection between the forward and/or reverse application of energy and/or motion by pushing forward and/or pulling back on the orthopedic impactor. During use, a user (e.g., a medical professional, a doctor, a surgeon, etc.) may push forward on an orthopedic impactor thereby causing a hammer of the impactor to strike a first and/or a forward impaction surface causing the orthopedic impactor to drive an orthopedic implant and/or surgical tool. Alternatively, or in addition, the user may pull back on the orthopedic impactor thereby causing the impactor's hammer to strike a second and/or a reverse impaction surface causing the orthopedic impactor to produce a reverse impaction to, for example, remove an orthopedic implant or surgical tool.

It should be appreciated that while, for example, the orthopedic impactor may be described herein in connection with driving a broach into a patient's bone to, for example, prepare an intramedullary canal of the patient's bone, the current subject matter is not so limited, and the orthopedic impactor may be used in connection with any surgical tool and/or implant now known and/or hereafter developed. As such, the current subject matter should not be limited to any particular surgical tool, device, instrument, implant, and/or procedure unless explicitly claimed. The orthopedic impactor may be arranged and/or configured to apply a force, while minimizing the risk of injury to the patient and/or to the user's hands during use. Moreover, the orthopedic impactor may be configured to assist its user to deliver a force towards and/or away from a surgical area in, for example, but not limited to, a joint replacement procedure.

In some implementations, the current subject matter relates to an impactor that may incorporate electromagnetic devices and/or systems. The impactor may include an electromagnetic component having a stationary electromagnetic housing and a moving armature component. The stationary electromagnetic housing may include one or more coils. The coil(s) may be configured to receive an electric current. The armature component includes one or more magnets that may be configured to generate a magnetic field that may interact with the electric current applied to the coil(s) and trigger translation movement of the armature component.

The impactor may further include a striker body coupled to the armature component and an object, where the striker body may include a distal end. The impactor may also include a strike chamber having an interior and a distal strike wall disposed at a distal end of the strike chamber, and a spacer disposed at a proximal end of the strike chamber and opposite the distal strike wall.

In some implementations, an electromagnetic field may be generated as a result of application of the electric current to the coil. The generated electromagnetic field may force the armature component and the striker body to translate. Translation of the armature component causes the distal end of the striker body to strike at least one of the distal strike wall and the spacer.

In some implementations, the directions of translation movement of the armature component and the striker body may be interdependent, which in turn, may be dependent on a direction of the current applied to the coil(s) within the stationary electromagnetic housing. Further, the direction of translation movement of the armature component and the striker body may be directly and/or indirectly dependent on the direction of the current. Further, a change in the direction of the current applied to the armature component may change the direction of translation movement of the armature component and the striker component.

FIG. 1 illustrates an exemplary impactor 100 that may be configured to incorporate a system for detecting whether a forward or a reverse impact is desired and, as a result, bidirectionally operating the impactor (as shown and described below), according to some implementations of the current subject matter. The impactor 100 may be configured to include a housing 102, a handle 104, an actuator or a trigger assembly 106, a power source 108, a distal connector assembly 110, a strike mechanism housing 112, a distal connector housing 114, electronics 116, and a strike mechanism 118 (positioned within the strike mechanism housing 112). In some example, non-limiting implementations, the impactor 100 may also include a user interface, an electronic display and/or an input keypad and/or key panel (e.g., touch-based keypad/key panel) that may be used to display and/or allow the user to enter various operational parameters of the impactor 100.

The housing 102 may be configured to house and/or enclose one or more components of the impactor 100. The housing 102 may be manufactured from any suitable material now known or hereafter developed such as, for example, but not limited to, plastic, metal, composite material, fiberglass, and/or any combination thereof.

The housing 102 may include the handle portion 104 with an optional handgrip for comfortable and secure holding of the impactor 100 for use during a procedure (e.g., positioning of an implant into a bone). Alternatively, or in addition, the housing 102 may incorporate a suitable mounting interface for integrating the impactor 100 into a robotic assembly during use. In some example implementations, the housing 102 may be a unitary structure and/or may include multiple components that may be assembled together.

The housing 102 may also include a reception port for receiving the power source or the battery (hereinafter, “battery”) 108. The battery 108 may be a rechargeable battery and may be removed from the housing 102 after use, such as, for example, for recharging. As can be understood, the battery 108 may recharged while coupled to the housing 102. Use of the battery 108 may provide for portability and versatility of the impactor 100 during use, i.e., the user of the impactor 100 does not have to be concerned with power wires (and/or pneumatic tubes) extending from the impactor 100. Alternatively, or in addition, the housing 102 may include one or more power ports (not shown in FIG. 1) that may be used to couple one or more power wires (e.g., to provide power and/or power in addition to the battery power 108) and/or one or more pneumatic tubes (e.g., to provide additional air pressure to the impactor 100 during use). As can be understood, more than one battery 108 may be included in the housing 102 and/or used during procedures. Any type of battery may be used, such as, for example, but not limited to, alkaline, nickel metal hydride (NiMH), lithium ion, and/or any combination thereof. The battery 108 may be incorporated and/or inserted into the handle 104 and/or coupled to the handle 104 (FIG. 1 illustrates the battery 108 being incorporated and/or inserted into the handle 104). As can be understood, other ways of coupling and/or connecting the battery 108 to electrical circuits of the impactor 100 are possible.

In some example, non-limiting implementations, the battery 108 may be at least one of the following: a capacitor, a supercapacitor, an interface for receiving mains power, an external power source, a chemical power source, and/or any combinations thereof. Further, combining a battery and/or a capacitor/supercapacitor may enable provision of a large power source that may, via charging of a capacitor, provide a large instantaneous and/or short-term current.

The battery 108 may be configured to provide current to power the electronics 116, which may be used to control operation of the impactor 100. The electronics 116 may be disposed in the handle 102, the housing 112, and/or both of the impactor 100. The battery 108 may be coupled to the electronics 116 using one or more wires. Alternatively, or in addition, the battery 108 may wirelessly (e.g., using near field) supply to the electronics 116. The electronics 116 may include a central processing unit (CPU) that may be arranged and configured to generate and/or execute one or more instructions associated with operation of the impactor 100. The electronics 116 may also include a memory for storing various data, information, etc., such as, for example, instructions for the CPU, a permanent storage for storing larger amounts of information, a communications interface for communicating with one or more components of the impactor 100 or with one or more external devices (e.g., a USB type port, a network connector, etc.), and/or any other components. In some example, alternate implementations, the impactor 100 and/or the electronics 116 may include one or more microcontrollers that may be arranged and configured to control and/or coordinate one or more components of the impactor 100.

In some implementations, as stated above, the impactor 100 may include one or more user interfaces (not shown in FIG. 1) that may be used for inputting various operational parameters and/or changes thereof. For example, the parameters may include, but are not limited to, a speed of an impact, a force of the impact, a frequency of the impact, a direction of the impact (e.g., whether to drive in an implant or remove an implant), a depth of the implant (e.g., how far into a bone the implant is to be driven), a stability of the implant (e.g., as may be measured by hoop stress), and/or any other parameters. These parameters may relate to a single impact and/or to multiple impacts. In some implementations, the user may input a single parameter, using which, the electronics 116 may be configured to determine any other operations parameters (e.g., input of a force parameter may lead the electronics determine that positioning of an implant is intended and should be performed at a certain speed).

In some implementations, the user interface may be used for not only inputting operational parameters of the impactor 100, but also for observing its operation and viewing how changes in one or more operational parameters may affect operation of the impactor 100. For example, a change in frequency of the impact may trigger changes (e.g., automatic changes and/or a request for manual changes) in force of the impact and a speed of the impact. The electronics 116 may determine such changes and display changed parameters, along with an any effects on the operation of the impactor 100 and/or implantation/removal process, on the user interface. For example, the effects may include an indication of a change in the remaining battery level, and/or a determined impact stability, a total impact energy that has been transferred to the implant, etc.

In some example, non-limiting implementations, the strike mechanism 118 may be used to actuate positioning and/or removal of an implant by the impactor 100 after receiving one or more instructions from the electronics 116. For example, upon actuating the trigger 106 (e.g., after entry of appropriate operational parameters), the electronics 116 may be configured to generate one or more operational instructions in accordance with one or more operational parameters and provide such instructions to the strike mechanism 118. Upon receiving instructions from the electronics 116, the strike mechanism 118 may be configured to impart force to the distal connector 110 (positioned within the housing 114), which, in turn, may transfer that force to an object (e.g., an implant, a tool, etc.) that may be coupled to the distal connector 110 and/or directly to the implant (either for positioning or removal).

In some implementations, the distal connector 110 may include one or more attachment structures and/or mechanism 120 that may be arranged and configured to couple to an object (e.g., drills, cutting tools, effectors, broaches, implants, etc.). For coupling to the distal connector 110, the object (not shown in FIG. 1) may include a click-fit, snap-fit, etc. attachment structure into which the distal connector 110 may mate with. Alternatively, or in addition, the distal connector may include one or more of the following attachment structures and/or mechanisms: a gripping structure (e.g., a clamp), a snap-fit structure, an interference-fit structure, a magnetic attachment mechanism, a releasable attachment mechanism, an interchangeable attachment structure/mechanism, and/or any other type of structures/mechanisms. In some implementations, the distal connector 110 may include a single attachment structure/mechanism and/or multiple attachment structures/mechanism, such as, for example, a snap-fit structure that may be used when implanting an object and a gripping structure that may be used for removing an object. The distal connector 110 may be arranged and configured to be used with a plurality of attachment structures/mechanism may also be beneficial since manufacturers of different implants may require different attachment structure.

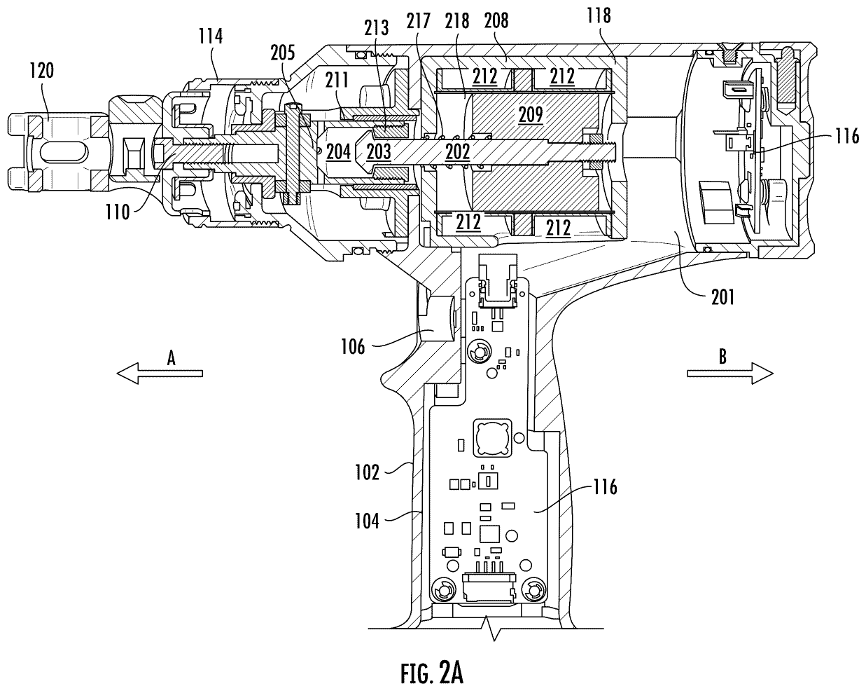

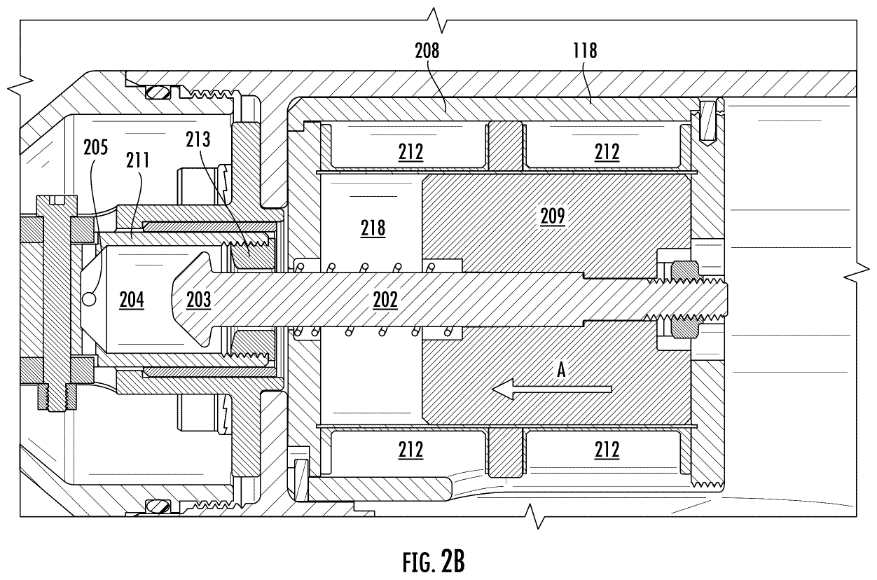

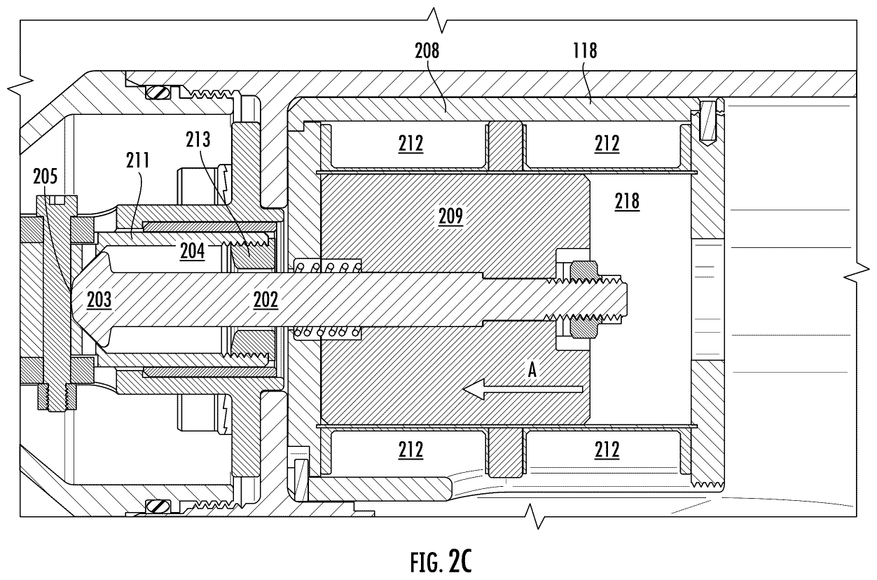

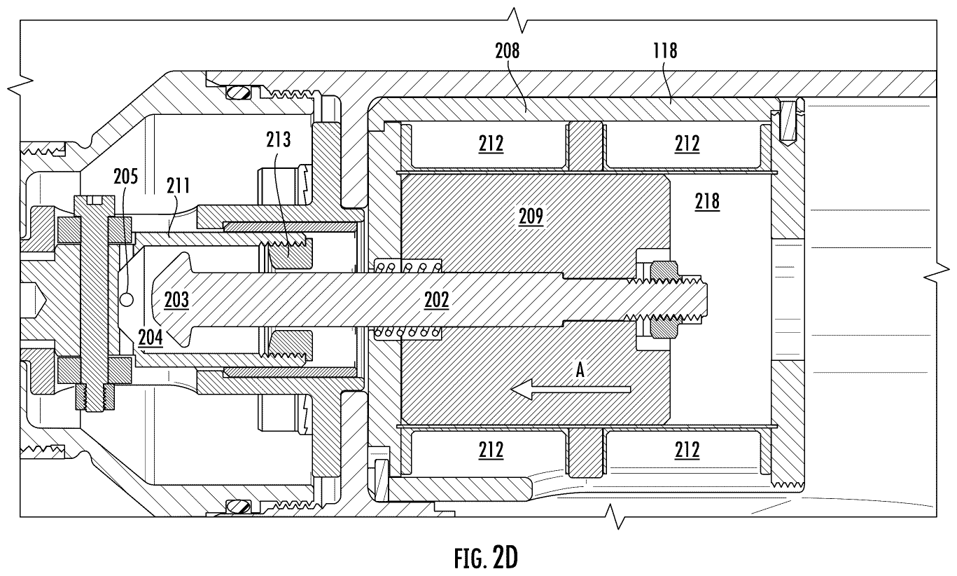

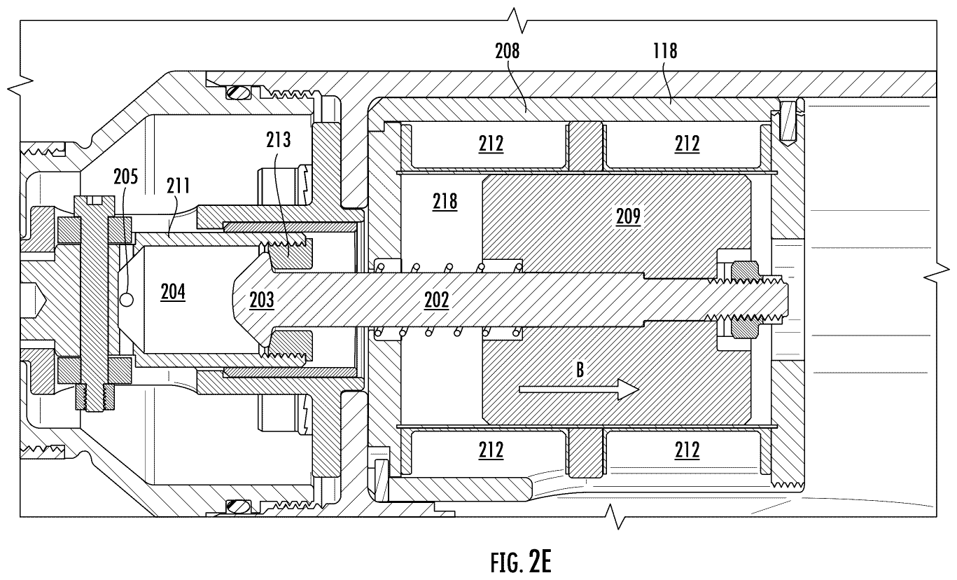

FIGS. 2a-e illustrate further details of the impactor 100 shown in FIG. 1. In particular, FIG. 2a is a cross-sectional view of the impactor 100 shown in FIG. 1, according to some implementations of the current subject matter. The cross-sectional view is taken along sectioning plane A-A shown in FIG. 1 and illustrates the details of the strike mechanism 118. FIGS. 2b-2e are perspective views of operation of the strike mechanism 118.

As stated above, the strike mechanism 118 may be used to interact with the object coupled to the distal connector 110 (e.g., using structure(s)/mechanism(s) 120) for the purposes of imparting force to the object. The force may, for example, be used to insert an implant into a bone of a patient, and/or to remove an implant from the bone (e.g., to replace it, to correct improper installation, etc.). As can be understood, the force imparted by the strike mechanism 118 may be used for any other purposes. The strike mechanism 118 may be configured to impart force as a result of receiving current signals from electronics 116. The current signals may be used to generate an electromagnetic field triggering movement of at least one component of the striker mechanism 118, thereby causing the distal connector 110 to translate.

As shown in FIG. 2a, the strike mechanism 118 may be disposed within an interior portion 201 of the actuator mechanism housing 112. The interior portion 201 may be at least partially hollow so that it may accommodate positioning of the strike mechanism 118. The strike mechanism 118 may include a striker body 202, a strike chamber 204, a stationary electromagnetic housing 208, an armature component 209 secured to the striker body 202, one or more coils 212 that may be positioned around the armature 209 and may be configured to interact with one or more permanent magnets disposed within the armature component 209.

The stationary electromagnetic housing 208 may be configured to remain stationary during operation of the impactor 100 while the armature component 209, along with the striker body 202, moves within an interior of the stationary electromagnetic housing 208 as a result of application of current. During operation of the impactor 100, the coil(s) 212 may receive electrical current from a battery (e.g., the power source 108, as shown in FIG. 1). In some example implementations, the housing 208 may circumferentially surround the armature component 209 as well as the coil(s) 212 and the striker body 202.

The housing 208 may also form a hollow channel 218 that may accommodate positioning and translational movement of the armature component 209 while being secured to the striker body 202. The armature component 209 may translate within the hollow channel 218 between strikes performed by the striker body 202 after application of electrical current to the coil(s) 212. As stated above, the armature component 209 may include one or more magnets, such as, for example, permanent magnets. As can be understood, the magnets may be any other types of magnets. The magnets may be disposed in pairs, where each pair of magnets may have opposite polarities. For instance, some magnets may have a north polarity while other magnets may have a south polarity. Any arrangement of magnets' polarities may be possible.

Each magnet may have a circular shape with an open interior portion (e.g., which may be similar to magnets having a donut shape). Outer diameters of the magnets may be the same and/or different. Similarly, the inner diameters of open interior portions of the magnets may be the same and/or different. In some example, implementations, by having the magnets have same outer diameters and same inner diameters of their respective interior portions may allow for a smoother translation movements of the armature component 209 within channel 218. The magnet polarities' arrangement may be configured to generate a high magnetic potential.

Alternatively, or in addition, no permanent magnets are used in the impactor 100. Instead, the armature component 209 may be ferromagnetic (e.g., 430FR stainless steel, and/or any other material) and/or may include one or more portions that are manufactured from ferromagnetic materials. Ferromagnetic materials may refer to materials that exhibit strong magnetic properties and may be characterized by an alignment of magnetic moments in a common direction. When the materials are exposed to an external magnetic field, they become magnetized and maintain such magnetization after removal of the field. By way of a non-limiting examples, ferromagnetic materials that may be used for the armature component 209 and/or any of its portions may include iron, cobalt, nickel, their alloys, and/or any other materials, and/or any combinations thereof. Use of ferromagnetic materials for and/or in the armature component 209 may allow the armature component 209 to interact with the current supplied to the coil(s) 212 without the use of permanent magnets. This may reduce an overall weight of the armature component 209 and thus, the impactor 100.

Once current is supplied to the coil(s) 212, the coil(s) 212 may become energized. The energized coil(s) 212 may interact with the magnetic potential forcing translation of the armature component 209 to translate within the channel 218. The direction of translation of the armature component 209 may depend on the direction of the current supplied to the coil(s) 212. By way of a non-limiting example, supplying the current in a counterclockwise direction in the coil(s) 212 may cause a forward translational movement of the armature component 209 causing the striker body 202 and connector 110 to translate forward (i.e., in direction A), and supply of the current in a clockwise direction in the coil(s) 212 may cause a reverse translational movement of the armature component 209 (i.e., in direction B). As can be understood, the current subject matter is not limited to the above examples, and the current may be supplied to the coil(s) 212 in any desired direction so that the armature component 209 may be arranged and configured to move in a particular desired direction. Further, the “forward” and “reverse” designations are non-limiting and are provided herein for illustrative purposes only.

The coil(s) 212 may be arranged and configured to be positioned proximate to the armature component 209 and may further be configured to surround the armature 209. Having the coil(s) 212 positioned proximate to the armature component 209 may allow for generation of an increased electromagnetic field, which, in turn, may allow for a more forceful translational movement of the armature component 209 (either forward or in reverse) as well as the striker body 202 and distal connector 110.

The striker component 202 and, in particular, its distal end 203 may be configured to translate within the strike chamber 204 upon application of electromagnetic force. The strike chamber 204 may be configured coupled to the distal connector 110 and may be positioned and translate within a passageway 211. Once the electromagnetic force is generated as a result of an application current to the coil(s) 212, the armature component 209 and the striker body 202 may be configured to translate within the strike chamber 204 causing the distal end 203 of the striker body 202 to come in contact with an interior strike wall 205 of the strike chamber 204, which is disposed at the distal end of the strike chamber 204. Upon the striker body 202 striking the interior strike wall 205, the strike chamber 204 may be configured to translate within the passageway 211, thereby translating the impact to any objects, tools, implants, etc. that may be coupled to the distal connector 110. Once impact motion is completed, a spring 217 that may be positioned and/or wrapped around the striker body 202 may assist with returning the striker body 202 and the armature component 209 to a pre-impact position.

In some implementations, the strike chamber 204 may also include a spacer 213 that may be disposed at a proximal end of the strike chamber 204 and opposite the strike wall 205. The spacer 213 may interact with the distal end 203 of the striker body 202 upon reverse translation of the striker body 202 within the strike chamber 204 and the housing 208. The reverse translation (i.e., direction B) may be caused by application of current to the coil(s) 212 in a direction that may be opposite to the direction of the current applied to the coil(s) 212 to cause the striker body 202 to translate in the forward direction (i.e., direction A). Alternatively, or in addition, the reverse translation may be caused during a recoil by the striker body 202 after a forward translation by the striker body 202 has been performed.

The strike chamber 204 may include one or more threads at its proximal end. The spacer 213 (as shown in FIGS. 3a-b) may also include threads that may be configured to interact with the threads of the strike chamber 204, thereby allowing the spacer to be threaded at the proximal end of the strike chamber 204, and thus, secured to the strike chamber 204.

In some implementations, the arrangement of the strike chamber 204 and the spacer 213 may allow for a reduction of undesired translations that may be in a direction that is opposite of the initial direction of translation. For example, during an intended forward impact (e.g., insertion of an implant, etc.) by the impactor 100, the current applied to the coil(s) 212 may cause the striker body 202 to translate forward, i.e., in the direction A, causing the distal end 203 of the striker body 202 to forcefully impact strike wall 205 of the striker chamber 204. The impact by the distal end 203 may cause the strike chamber 204 and the distal connector 110 may be configured to move forward, i.e., in the direction A. Once impact occurs, a reverse translation, i.e., in the direction B, by the striker body 202 may occur, which may cause a pull back, and, thus, a partial reduction of the intended impact (e.g., a partial dislodgement of the inserted implant). Similarly, during an intended reverse impact (e.g., removal of an implant, etc.) by the impactor 100, the current applied to the coil(s) 212 (in an opposite direction) may cause the striker body 202 to translate in reverse, i.e., in the direction B, causing the distal end 203 of the striker body 202 to forcefully impact the spacer 213. The impact by the distal end 203 may cause the strike chamber 204 and the distal connector 110 to move in reverse, i.e., in the direction B. Once the distal end 203 impacts the spacer 213, a forward translation, i.e., in the direction A, of the striker body 202 may also occur. This may produce an opposite effect, e.g., a partial dislodgement of the inserted implant. To prevent that, the spacer 213, having a predetermined width, may be positioned at the proximal end of the strike chamber 204, thereby reducing the distance that the distal end 203 of the striker body 202 may translate within the strike chamber 204, and thus, reducing or minimizing the effect of such unintended opposite direction translations.

In some implementations, the impactor 100 may be configured (through at least use of the arrangement of the striker body 202, the strike chamber 204 and the spacer 203) to generate a high energy impulse output movement (either forward or reverse) to allow for the impactor to deliver a high energy impulse to the output of the device in both forward and reverse impact. If the impactor 100 detects that a single directional impulse is desired (such as for example, but not limited to, using a selection setting), then the impactor 100 may be configured to determine whether a forward impulse and/or a reverse impulse is desired, such as, for example, through a selection setting, use of one or more sensors (e.g., positioned in the distance connector 110, armature component 209, electronics 116, and/or anywhere in the impactor 100), and/or by detecting whether the user is pressing (e.g., to insert an implant) and/or pulling (to remove an implant) on the impactor 100 in relation to the surgical site. For instance, upon determining that the user is pressing the impactor 100 on the surgical site, the electronics 116 of the impactor 100 may determine that a forward impact (e.g., in the direction A) is desired. The impactor electronics 116 may then deliver a high (e.g., a maximum) current from the impactor's power source 108 to the coil(s) 212 in a first direction, which, in turn, may translate the striker body 202 forward causing its distal end 203 to impact the impact strike wall 205, thereby translating the strike chamber 204 and the distal connector 110 to translate forward (i.e., in the direction A). Once impact occurs, the electronics 116 may immediately and/or after a predetermined period of time (e.g., one millisecond, few milliseconds, etc.) deliver a low (e.g., minimum) current from the impactor's power source 108 to the coil(s) 212 in a second direction. This may slow the recoil of the striker body 202 after the impact, thereby preventing reduction of the effect of the impact produced by the initial forward translation of the strike chamber 204 and the distal connector 110. Similar operation may occur when the impactor detects a pulling movement, such as, for example, when the user wishes to remove an implant from the surgical site. Alternatively, or in addition, to prevent reduction of initial impacts (either in forward or reverse direction), a spring (e.g., spring 217) may be used to reduce translational movement that is opposite to the initial impact.

In some implementations, subsequent (e.g., immediately thereafter, a short time after (e.g., 1 millisecond, few milliseconds, etc.), etc.) to the high-impulse forward impact operation, the electronics 116 may also be configured to deliver low (e.g., minimum) current to the coil(s) 212 to counteract effects of a spring that causes return of the armature component 209. Such spring-based return may be counter-effective to the delivered forward impact, and, as discussed herein, may, for example, cause unseating of an acetabular cup that was being set using the forward impact. The low current can counteract the return force produced by the spring by slowing down return movement (i.e., opposite of the forward impact movement) of the armature component 209, and thus, reduce the possibility of unseating of the cup. The current may be delivered to the coil(s) 212 in the same or opposite direction as the direction of the current for the high-impulse forward impact. As can be understood, application of such low current to counter effects of a return of the armature component 209 after an impact may be during the forward impact and/or reverse impact produced by the impactor 100.

In some examples, one or more sensors positioned, for example, in the housing 208, armature component 209, and/or anywhere else in the impactor 100, may be configured to determine position of the armature component 209 prior to movement for delivery of an impact, during movement for delivery of the impact, during movement after delivery of the impact, and/or after movement. The sensors can be any type of sensors, such as, for example, but not limited to, position sensors, pressure sensors, infrared sensors, and/or any other type of sensors, and/or any combinations thereof. The armature component 209's position data gathered by the sensors may be provided to the electronics 116. The electronics 116 may use the position data to determine location of the armature component 209 within the housing 208, and, based on the location, determine an amount of current that may need to be supplied to the coil(s) to affect movement of the armature component 209. For example, the sensors may determine that a forward impact has just occurred and that the armature component 209 is moving in a return direction. This may cause the electronics 116 to determine that a low current may need to be applied to the coil(s) 212 to slow the return movement of the armature component 209. In some example implementations, the sensors may be configured to detect, interrogate, and/or determine armature component's position data continuously, periodically, during a specific movement of the armature component, dynamically (e.g., upon initiation of impact-related operations by the electronics 116), upon request, and/or using any other desired schedule.

FIGS. 3a-b are perspective views of the spacer 213 that may be disposed in the proximal end of the strike chamber 204, according to some implementations of the current subject matter. The spacer 213 may be configured to include a substantially circular profile having a threaded body 302 disposed between a proximal end 304 and a distal end 306. The proximal end 304 may be configured to include a ledge 308. The proximal end 304 may include an opening 310. The distal end 306 may include an opening 312. A substantially cylindrical interior channel 314 may be disposed between the proximal end 304 and the distal end 306 connecting the opening 310 and the opening 312.

The threaded body 302 may include one or more threads 318 disposed on an exterior of the threaded body 302. The threads 318 may be configured to interact with one or more threads disposed on an interior wall of the strike chamber 204 (not shown in FIGS. 3a-b) during insertion or removal of the spacer 213 into or from the strike chamber 204. For example, upon insertion of the spacer 213 into the strike chamber 204, the threads 318 may be used to thread (e.g., in a clockwise direction) the spacer 213 into the strike chamber 204 until the ledge 308 meets and/or is adjacent to the proximal end of the strike chamber 204, thereby preventing further threading of the spacer 213 into the strike chamber 204. To remove the spacer 213 from the strike chamber 204, the threads 318 may be used to un-thread (e.g., in a counterclockwise direction) the spacer 213 from the strike chamber 204.

Upon positioning of the spacer 213 into the strike chamber 204, the striker body 202 may be positioned in the channel 314 of the spacer 213. The channel 314 may be sized to allow for insertion and, thus, translation of the striker body 202 (not shown in FIGS. 3a-b). For example, a diameter (and/or any other dimensions) of the channel 314 may be slightly larger than the diameter (and/or any other corresponding dimensions) of the striker body 202. The striker body 202 may be inserted into the channel 314 via an opening 316 in the sidewall of the threaded body 302 once the spacer 213 is positioned in the strike chamber 204 (not shown in FIGS. 3a-b). Once the striker body 202 is positioned into the channel 314, it may be configured to translate in the channel 314 upon application of current to the coil(s) 212.

FIGS. 2b-e illustrate operations of the strike mechanism 118, according to some implementations of the current subject matter. In particular, FIGS. 2b-d illustrate a forward impact operation and FIG. 2e illustrates a reverse impact operation.

Referring to FIGS. 2b-d, to initiate the forward impact operation, the impactor's electronics 116 (not shown in FIGS. 2b-d) may be configured to detect that the user of the impactor is pushing on the object, tool, implant, etc. coupled to the distal connector 110. Upon such detection, the electronics 116 may deliver current to coil(s) 212 positioned around the striker body 202, which may generate an electromagnetic force. The electromagnetic force may cause the armature component 209 to move toward the distal end of the housing 208 (e.g., in the direction A) within the hollow channel 218. Movement of the armature component 209 may trigger movement of the striker body 202 toward the distal end 205 of the strike chamber 204. The striker body 202 may move within the strike chamber 204 until it impacts the strike wall 205, as shown in FIG. 2c. The striker body 202 impacting the strike wall 205 may also deliver force to the strike wall 205, which may be proportional (directly and/or indirectly) to the electromagnetic force generated by the current delivered to the coil(s) 212.

As shown in FIG. 2d, delivery of the force to the strike wall 205 may cause the strike chamber 204 and the distal connector 110 (not shown in FIG. 2d) to translate forward (in the direction A). Such forward movement of the strike chamber 204 may also move it away from the distal end of the housing 118. This movement may transfer the delivered force to the object, tool, implant, etc. that may be coupled to the distal connector 110. The striker body 202 may temporarily remain in place (as shown in FIG. 2d) while translation of the strike chamber 204 is occurring. Further, the striker body 202 may remain in place as a result of the electromagnetic force that has been applied to generate the forward movement. Once the electromagnetic force is no longer present, the spring 217 may return the striker body 202 and the armature component 209 may return to its pre-impact position.

In some example implementations, to reduce the effect of an opposite direction movement subject to the forward impact, the electronics 116 (not shown in FIG. 2d) may be configured to apply current to the coil(s) 212. The applied current may be smaller than the current used to generate the initial forward impact. The current may be in the same or opposite direction to the current used for the initial impact. Application of the current may be for a short period of time, e.g., 1 millisecond, few milliseconds, etc. Such application may generate a smaller magnitude electromagnetic force that may slow translation of the striker body 202 in the direction that is opposite to direction A. This may prevent the striker body 202's distal end 203 from forcefully impacting the spacer 213 on a return and thus, translating the strike chamber 204 and the distal connector 110 in a reverse direction, thereby reducing or preventing opposite movement of the object, tool, implant, etc. coupled to the distal connector 110.

FIG. 2e illustrates a reverse impact operation of the impactor 100, according to some implementations of the current subject matter. The reverse impact operation may be performed when, for example, a removal of an implant, tool, object, etc. may be desired. To initiate the reverse impact operation, the impactor's electronics 116 (not shown in FIGS. 2b-d) may be configured to detect that the user of the impactor is pulling on the object, tool, implant, etc. coupled to the distal connector 110. Upon such detection, the electronics 116 may deliver current to coil(s) 212 (e.g., in a reverse direction to the direction of the current delivered for the forward impact operation) positioned around the striker body 202. Delivery of the current may generate an electromagnetic force that may cause the armature component 209 to move away from the distal end of the housing 208 (e.g., in the direction B) within the hollow channel 218. Movement of the armature component 209 may trigger movement of the striker body 202 toward the spacer 213 disposed within the strike chamber 204. The striker body 202 may move within the strike chamber 204 until it impacts the spacer 213, as shown in FIG. 2e. The striker body 202 impacting the strike wall 205 may also deliver force to the spacer 213. Similar to the forward impact operation, the force may be proportional (directly and/or indirectly) to the electromagnetic force generated by the current delivered to the coil(s) 212.

The delivered force to the spacer 213 may cause the strike chamber 204 and the distal connector 110 (not shown in FIG. 2e) to translate in reverse (in the direction B). Such reverse movement of the strike chamber 204 may also move the distal connector 110 along with the object, tool, implant, etc. that may be coupled to the distal connector 110. This may, for instance, cause dislodgement of an implant that may be positioned in the bone of a patient. Similar to the forward impact operation, the striker body 202 may temporarily remain in place while reverse translation of the strike chamber 204 is occurring. Further, the striker body 202 may remain in place as a result of the electromagnetic force that has been applied to generate the reverse movement.

In some example implementations, to reduce the effect of an opposite direction movement (e.g., forward direction movement), the electronics 116 (not shown in FIG. 2e) may be configured to apply current to the coil(s) 212, which may be smaller than and may be in the same or opposite direction to the current used to generate the initial reverse impact. Application of the current may be for a short period of time, e.g., 1 millisecond, few milliseconds, etc. Such application may generate a smaller magnitude electromagnetic force that may slow translation of the striker body 202 in the direction that is opposite to direction B. This may prevent the striker body 202's distal end 203 from forcefully impacting the strike wall 205 and thus, translating the strike chamber 204 and the distal connector 110 in the forward direction, which may, for instance, cause re-insertion of the implant positioned in the bone that the user may be trying to remove.

In some implementations, the impactor 100 may be operated in various modes. For example, the impactor 100 may operate in a single forward or reverse impact mode, where delivery of a single impact in one direction may be desired. In this case, the electronics 116 of the impactor 100 may be configured to deliver a higher current for a such single impact and, optionally, a smaller current to prevent translation of the distal connector 110 in an opposite direction. Alternatively, or in addition, opposite direction movement may be controlled by a spring (e.g., spring 217).

In another example mode, e.g., a “broach mode,” the impactor 100 may be operable to deliver multiple forward or reverse impacts. In this case, the electronics 116 may be configured to deliver multiple current pulses for generation of intermittent applications of the electromagnetic force for movement of the armature component 209 and the striker body 202. Higher current pulses may be delivered for the purposes of intended impacts (e.g., forward impacts) and smaller current pulses (in the same or opposite direction) may be delivered to counteract translations of the striker body 202 in an opposite direction to the directions of the intended impacts. Such smaller current pulses may be delivered either immediately and/or a predetermined period of time (e.g., 1 millisecond, few milliseconds, etc.) after each higher current pulse. Also, the smaller current pulses may be delivered for a short duration (e.g., 1 millisecond, few milliseconds, etc.) to ensure that the continuous operation of the impactor 100 in the intended impact direction.

The following is a discussion of an exemplary, non-limiting orthopedic surgical instrument and/or impactor that may be used in connection with impactor 100 shown in FIG. 1. As can be understood, the current subject matter is not limited to the use implementation of the impactor and/or any specific tools, and may be used in connection with any desired devices, mechanisms, tools, etc.

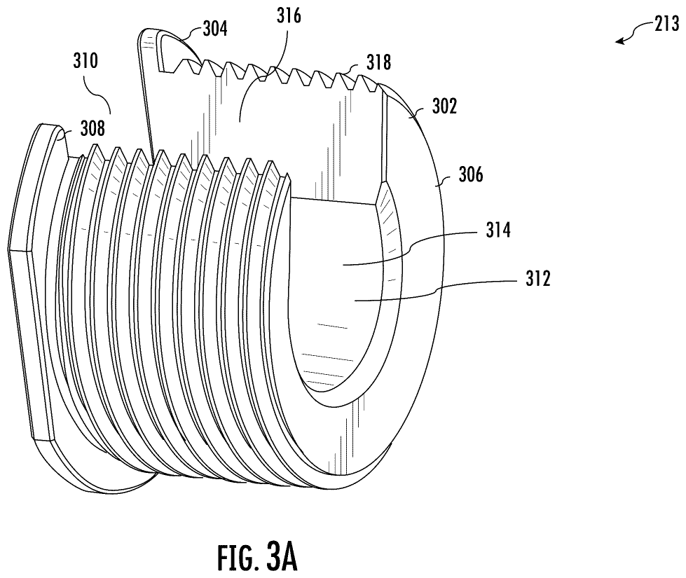

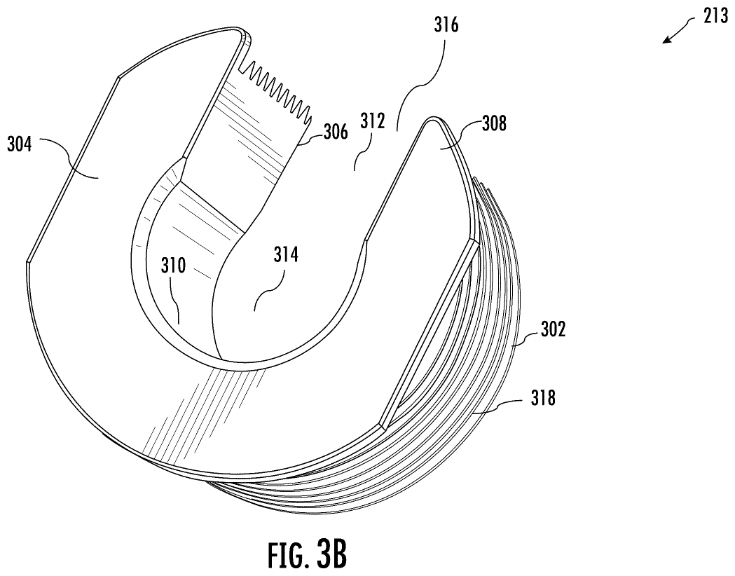

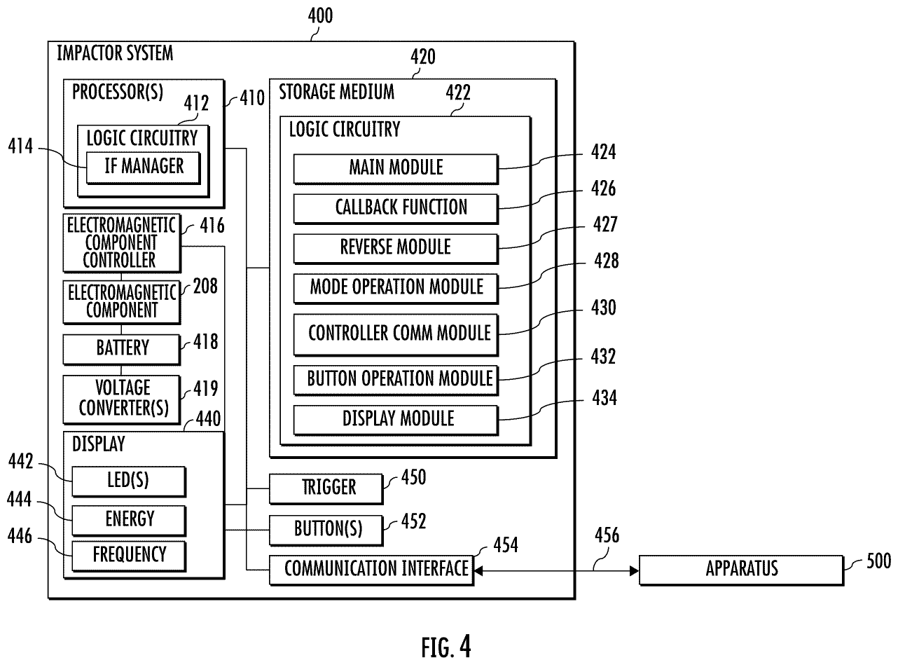

FIG. 4 is a block diagram of an exemplary orthopedic surgical instrument and/or impactor 400, according to some implementations of the current subject matter. The impactor 400 may be similar to the impactor shown and described above in connection with FIGS. 1-3.

The impactor 400 may combine with any suitable example of the systems, devices, and methods disclosed herein. The impactor 400 may include processor(s) 410, a non-transitory storage medium 420, an electromagnetic component controller 416, a battery 418, a voltage converter 419, a display 440, a trigger 450, button(s) 452, and a communication interface 454. The processor(s) 410 may include one or more processors, such as a programmable processor, a micro-controller unit (MCU), and/or the like. The processor(s) 410 may include processing circuitry to implement impactor logic circuitry 412 and 422.

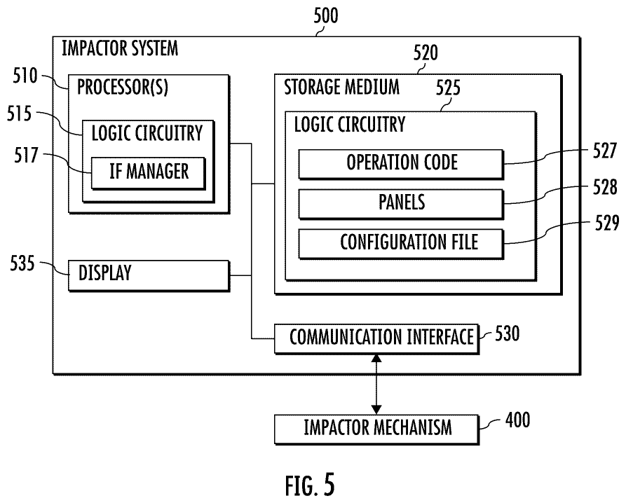

The processor(s) 410 may operatively couple with a non-transitory storage medium 420. The non-transitory storage medium 420 may store logic, code, and/or program instructions executable by the processor(s) 410 for performing one or more operations including the operations of the impactor logic circuitry 422. The non-transitory storage medium 420 may include one or more memory units (e.g., fixed or removable media or external storage such as a flash memory, secure digital (SD) card, random-access memory (RAM), read only memory (ROM), a flash drive, a hard drive, a solid-state drive (SSD) and/or the like). The memory units of the non-transitory storage medium 420 can store logic, code and/or program instructions executable by the processor(s) 410 to perform any suitable implementations of the current subject matter, as described herein. For example, the processor(s) 410 may execute instructions such as instructions of impactor logic circuitry 412 causing the electromagnetic component 208 to operate the impactor at an impact energy and/or frequency selected by a user via button(s) 452 and/or via apparatus 500 (as shown in FIG. 5).

The processor(s) 410 may include code for the impactor 400 in memory within the processor(s) 410 and/or closely connected such as flash memory. The impactor logic circuitry 412 may represent code in or near the processor(s) 412 for execution by the processor(s) 410 and may include a user interface manager 414. The user interface manager 414 may include code executing on the processor(s) 410 to detect and respond to user input as well as to detect the electromagnetic component controller 416 (such as, for example, a Maxon EPOS4 Controller) and establish communication with the electromagnetic component controller 416.

The user interface manager 414 may communicate with the electromagnetic component controller 416 to receive status information about the electromagnetic component 208 and to control operation of the electromagnetic component 208. For instance, all button presses of button(s) 452 and edit events may be posted to the user interface manager 414 and processed in real-time. The user interface manager 414 may communicate commands with the electromagnetic component controller 416 to execute in response to the user's actions via button presses, system states, and error conditions. The user interface manager 414 may communicate alerts, warnings, and notifications to a user via the display 440 and or the apparatus 500 (as shown in FIG. 5) via the communications interface 454. Further, the user interface manager 414 may also handle user's response to alerts.

The battery 418 may include any desired power source.

The voltage converter(s) 419 may include a DC-DC voltage converters to adjust the voltage of signals to various voltages required to operate the components of the impactor 400 such as the processor(s) 410, the storage medium 420, and electromagnetic component controller 416, the display 440, the trigger 450, the buttons 452, the communications interface 454, and/or the like.

The storage medium 420 may include a code for execution by the processor(s) 410 to operate the impactor 400. If desired, the processor(s) 410 may copy code from the storage medium 420 to memory closer to the processor(s) 410 to facilitate faster execution of the code. For instance, the user interface manager 414 may include code copied from the impactor logic circuitry 422 to memory closer to the processor(s) 410 for execution.

The impactor logic circuitry 422 may include code for operation of the impactor 400 stored in hardware of the storage medium such as volatile or non-volatile memory in the storage medium 420. The impactor logic circuitry 422 may include a main module 424, a callback module 426, a reverse module 427, a mode operation module 428, a controller communications module 430, a button operation module 432, and a display module 434.

The main module 424 may include setup and loop functions. The setup function may run once at start-up and the loop function may run continuously afterwards. The setup function may attach interrupts that run when button(s) 452 are pressed on the user interface, initializes Timer1 which runs the trigger interrupt service routine (ISR), and initializes an impact delay for the electromagnetic component 208. The loop function allows the electromagnetic component 208 to operate in the user-desired mode when the trigger 450 is enabled and pulled. The loop function also handles showing the user that the trigger state is enabled via LED(s) 442 of the display 440 and/or via the apparatus 500 (shown in FIG. 5).

The callback function 426 may be, e.g., an ISR that runs every millisecond. In some example implementations, the callback function 426 may run periodically with at a time period of more than one millisecond or less than one millisecond.

The reverse module 427 may include functions to prepare to reverse the direction of translation of the electromagnetic component 208, direction change of the electromagnetic component 208, calculate impact delay of the impactor, and setup flutter time delays to set the frequency of impact while in flutter mode. These functions may switch the direction of translation of the electromagnetic component 208, reversing the electromagnetic component 208 to allow for bi-directional operation of the impactor, and may also determine the delay between reversals for controlling a frequency of impacts of the impactor in a flutter mode.

The mode operation module 428 may include the functions of position check, flutter check, and oscillation check functions which are called for normal/full-swing mode, high-frequency/flutter mode, and oscillation mode, respectively. Normal operation checks the position of the electromagnetic component 208 then calls the prepare to reverse function.

The controller communication module 430 may include the functions of enable electromagnetic component controller 416 functions, set current, set current direction, and disable the electromagnetic component controller 416 functions. These functions communicate to the electromagnetic component controller 416 whether or not to operate the electromagnetic component 208.

The button operation module 432 may include functions to handle setting user-desired frequency to operate the electromagnetic component 208 in addition to setting the operation mode and enabling the trigger 450. The functions may include energy plus to increase the energy of impact by the impactor, energy minus to increase the energy of impact by the impactor, frequency plus to increase the frequency of impacts by the impactor, frequency minus to decrease the frequency of impacts by the impactor, select operating mode to switch between available modes of operation (e.g., full-swing mode, flutter mode, or oscillation mode), and set trigger state to enable or disable the trigger 450. In some implementations, these functions may be accessed via the apparatus 500 (shown in FIG. 5) and/or the button(s) 452. In alternate implementations, a touch screen may be included in the display in lieu of or in addition to the button(s) 452.

The display module 434 may include functions handle the logic for displaying the amperage and frequency on the user interface. The functions may include energy display and frequency display.

The display 440 may include LED(s) 440 and numerical, alphanumeric, or graphical displays such as LED displays or liquid crystal displays (LCDs) to present a number representative of the energy 444 and frequency 446 selected for operation of the electromagnetic component 208. The button(s) 452 may include one or more buttons located in the display 440 and, in some implementations, adjacent to the energy 444 and frequency 446 displays to provide a user with an interface to increase and/or decrease the energy and/or frequency of the impact of the impactor on the forward and/or the reverse motion.

The trigger 450 may include a trigger or other button or switch that, when actuated, can cause the impactor 400 to operate if the trigger 450 is enabled. If the trigger 450 is disabled, depressing the trigger 450 may not cause the impactor 400 to operate. In some implementations, the trigger 450 cannot be depressed when the trigger 450 is disabled.

The processor(s) 410 may couple to a communication interface 454 to communicate with an apparatus 500 via a communications medium 456. The communications medium 456 may comprise a wired or wireless interface to communicatively coupled the impactor 400 with the apparatus 500 shown in FIG. 5.

The communication interface 454 may communicate user commands to and/or from the apparatus 500 to the impactor 400 to operate the impactor 400 via the functionality described in conjunction with the impactor 400. In some implementations, the apparatus 400 may operate the electromagnetic component 208 in addition to configuring parameters of operation of the electromagnetic component 208 such as the operating current, the upper frequency bound, the lower frequency bound, the operating frequency, the mode of operation of the electromagnetic component 208, and/or the like. In some implementations, the communication interface 454 may communicate information about the operation of the impactor 400 to the apparatus 500 such as the energy of operation, the frequency of operation, the mode of operation, events or alerts associated with the impactor 400, and log information such as time and date of use, impact detections, encoder counts, and/or the like.

The communication interface 456 (and similarly, communication interface 530 shown in FIG. 5) may include circuitry to transmit and receive communications through a wired and/or wireless media such as an Ethernet interface, a wireless fidelity (Wi-Fi) interface, a cellular data interface, and/or the like. In some implementations, the communication interface 456 (and/or interface 530) may implement logic such as code in a baseband processor to interact with a physical layer device to transmit and receive wireless communications from apparatus 500. For example, the communication interface 456 (and/or interface 530) may implement one or more of local area networks (LAN), wide area networks (WAN), infrared, radio, Wi-Fi, point-to-point (P2P) networks, telecommunication networks, cloud communication, and the like.

FIG. 5 illustrates an exemplary computing apparatus 500, according to some implementations of the current subject matter. The apparatus 500 may be a computing device that may be communicatively coupled with an orthopedic surgical instrument or impactor such as, orthopedic impactor 400 (e.g., as shown in FIG. 4). The apparatus 500 may be a computer in the form of a smart phone, a tablet, a notebook, a desktop computer, a workstation, or a server. The apparatus 500 can combine with any suitable example of the systems, devices, and methods disclosed herein. The apparatus 500 can include processor(s) 510, a non-transitory storage medium 520, communication interface 530, and a display 535. The processor(s) 510 may comprise one or more processors, such as a programmable processor (e.g., a central processing unit (CPU)). The processor(s) 510 may comprise processing circuitry to implement impactor logic circuitry 515 such as the impactor logic circuitry 412 shown in FIG. 4.

The processor(s) 510 may include memory such as flash memory to contain program code for execution by the processor(s) 510. In some implementations, the processor(s) 510 may have random access memory to contain a copy of code from flash memory or read only memory to facilitate faster execution of code. In some implementations, the processor(s) 510 may include cache to contain data for faster calculations or execution. In some implementations, the processor(s) 510 may include an impactor logic circuitry 515, which may include a user interface manager 517. The user interface manager 517 may function as a state machine controlled by keypad inputs, internal events or alarms, boundary conditions, exceptions, and supervisory input to the user interface manager 517. The user interface manager 517 may process button presses and may update a main screen on the display 535 reflecting the state of the application.

Upon startup of the user interface manager 517, a handler may be installed to detect the electromagnetic component controller 416 of the impactor 400 and to establish communication with the electromagnetic component controller 416. In some implementations, the button presses of button(s) 452 and edit events may be posted to a panel in the display 535 and may be processed in real-time. Controller commands may be executed upon the user's actions via button presses, system states, and error conditions. Further, the user interface manager 517 may implement alerts, warnings, and notifications and display the alerts, warnings, and notifications via the display 535. The user interface manager 517 may also include code to handle the user's response to alerts, warnings, and notifications.

The processor(s) 510 may operatively couple with a non-transitory storage medium 520. The non-transitory storage medium 520 may store logic, code, and/or program instructions executable by the processor(s) 510 for performing one or more instructions including the impactor logic circuitry 525. The non-transitory storage medium 520 may include one or more memory units (e.g., fixed and/or removable media or external storage such as electrically erasable programmable read only memory (EEPROM), a secure digital (SD) card, random-access memory (RAM), a flash drive, solid-state drive, a hard drive, and/or the like). The memory units of the non-transitory storage medium 520 may store logic, code and/or program instructions executable by the processor(s) 510 to perform any suitable implementation of the methods described herein. For example, the processor(s) 510 may execute instructions such as instructions of impactor logic circuitry 525 causing one or more processors of the processor(s) 510 to communicate user commands to an impactor 400 (as shown in FIG. 4a) and/or to communicate events, alerts, operation parameters for the impactor 400, and configurations.

The impactor logic circuitry 525 may include operation code 527, panels 528, and a configuration file 529. The operation code 527 may include functionality to set energy boundaries for operation of the impactor 400, set frequency boundaries for operation of the impactor 400, set an operating energy, set an operating frequency, set an impactor detection profile, set an operating mode (full swing, flutter, or oscillation), and/or the like.

The panels 528 may define graphical user interfaces for display of information and for receiving input parameters or configurations from a user. The configuration file 530 may include user selected parameters such as a controller with which to communicate, boundaries for energy (current), boundaries for frequency of impact, numbers of interrupts expected for push current and for pull current, and/or number of interrupts to receive to establish a frequency of impact.

The processor(s) 510 may couple to a communication interface 530 to transmit the data, code, or commands to and/or receive data, code, or commands from one or more external devices (e.g., a terminal, display device, a smart phone, a tablet, a server, or other remote device). The communication interface 530 includes circuitry to transmit and receive communications through a wired and/or wireless media such as an Ethernet interface, a wireless fidelity (Wi-Fi) interface, a Bluetooth interface such as a Bluetooth Low Energy (BLE) interface, a cellular data interface, and/or the like. In some implementations, the communication interface 530 may implement logic such as code in a baseband processor to interact with a physical layer device to transmit and receive wireless communications from the impactor 400. For example, the communication interface 530 may implement one or more of local area networks (LAN), wide area networks (WAN), infrared, radio, Bluetooth, Wi-Fi, point-to-point (P2P) networks, telecommunication networks, cloud communication, and the like.

The processor(s) 510 may couple to a display 530 to display panels 528 for a user interface and/or other user interface items such as a message or notification via, graphics, video, text, and/or the like. In some implementations, the display 530 may include a display on a terminal, a display device, a smart phone, a tablet, a server, or a remote device.

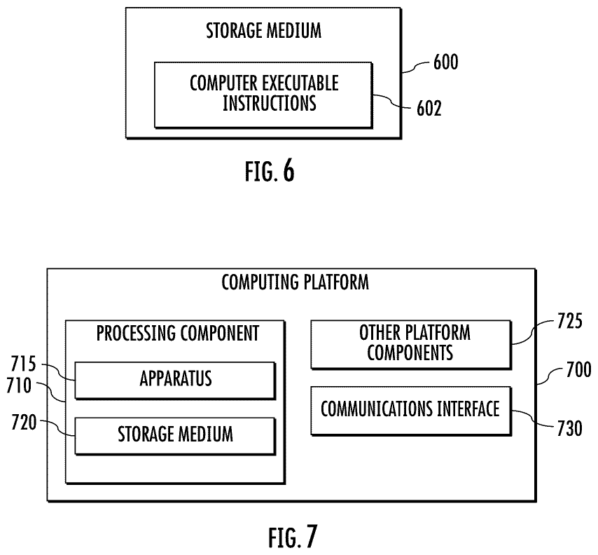

FIGS. 6-7 illustrate example implementations of a storage medium and computing platform for an orthopedic surgical instrument or impactor in accordance with one or more features of the present disclosure. FIG. 6 illustrates an example of a storage medium 600 to store impactor logic. Storage medium 600 may include an article of manufacture. In some examples, storage medium 600 may include any non-transitory computer readable medium or machine readable medium, such as an optical, magnetic or semiconductor storage. Storage medium 600 may store various types of computer executable instructions 602, such as instructions to implement logic flows and/or techniques described herein. Examples of a computer readable or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions may include any suitable type of code, such as source code, compiled code, interpreted code, executable code, static code, dynamic code, object-oriented code, visual code, and the like. The examples are not limited in this context.

FIG. 7 illustrates an example computing platform 700. In some examples, as shown in FIG. 7, the computing platform 700 may include a processing component 710, other platform components or a communications interface 730. According to some examples, computing platform 700 may be implemented in a computing device such as a server in a system such as a data center or server farm that supports a manager or controller for managing configurable computing resources as mentioned above. Further, the communications interface 730 may include a wake-up radio (WUR) and may be capable of waking up a main radio of the computing platform 700.

According to some examples, processing component 710 may execute processing operations or logic for apparatus 715 described herein such as the impactor logic circuitry 412, 515, and 525 illustrated in FIGS. 4-5. Processing component 710 may include various hardware elements, software elements, or a combination of both. Examples of hardware elements may include devices, logic devices, components, processors, microprocessors, circuits, processor circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements, which may reside in the storage medium 720, may include software components, programs, applications, computer programs, application programs, device drivers, system programs, software development programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an example is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given example.

In some examples, other platform components 725 may include common computing elements, such as one or more processors, multi-core processors, co-processors, memory units, chipsets, controllers, peripherals, interfaces, oscillators, timing devices, video cards, audio cards, multimedia input/output (I/O) components (e.g., digital displays), power supplies, and so forth. Examples of memory units may include without limitation various types of computer readable and machine readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory), solid state drives (SSD) and any other type of storage media suitable for storing information.

In some examples, communications interface 730 may include logic and/or features to support a communication interface. For these examples, communications interface 730 may include one or more communication interfaces that operate according to various communication protocols or standards to communicate over direct or network communication links. Direct communications may occur via use of communication protocols or standards described in one or more industry standards (including progenies and variants) such as those associated with the PCI Express specification. Network communications may occur via use of communication protocols or standards such as those described in one or more Ethernet standards promulgated by the Institute of Electrical and Electronics Engineers (IEEE). For example, one such Ethernet standard may include IEEE 802.3-2012, Carrier sense Multiple access with Collision Detection (CSMA/CD) Access Method and Physical Layer Specifications, Published in December 2012 (hereinafter “IEEE 802.3”). Network communication may also occur according to one or more OpenFlow specifications such as the OpenFlow Hardware Abstraction API Specification. Network communications may also occur according to Infiniband Architecture Specification, Volume 1, Release 1.3, published in March 2015 (“the Infiniband Architecture specification”).

Computing platform 700 may be part of a computing device that may be, for example, a server, a server array or server farm, a web server, a network server, an Internet server, a workstation, a mini-computer, a main frame computer, a supercomputer, a network appliance, a web appliance, a distributed computing system, multiprocessor systems, processor-based systems, or combination thereof. Accordingly, functions and/or specific configurations of computing platform 700 described herein, may be included or omitted in various implementations of computing platform 700, as suitably desired.

The components and features of computing platform 700 may be implemented using any combination of discrete circuitry, ASICs, logic gates and/or single chip architectures. Further, the features of computing platform 700 may be implemented using microcontrollers, programmable logic arrays and/or microprocessors or any combination of the foregoing where suitably appropriate. It is noted that hardware, firmware and/or software elements may be collectively or individually referred to herein as “logic.”

It should be appreciated that the exemplary computing platform 700 shown in the block diagram of FIG. 7 may represent one functionally descriptive example of many potential implementations. Accordingly, division, omission or inclusion of block functions depicted in the accompanying figures does not infer that the hardware components, circuits, software and/or elements for implementing these functions would necessarily be divided, omitted, or included in implementations.

One or more features of at least one example may be implemented by representative instructions stored on at least one machine-readable medium which represents various logic within the processor, which when read by a machine, computing device or system causes the machine, computing device, or system to fabricate logic to perform the techniques described herein. Such representations, known as “IP cores,” may be stored on a tangible, machine readable medium and supplied to various customers or manufacturing facilities to load into the fabrication machines that actually make the logic or processor.

The foregoing description has broad application. While the present disclosure refers to certain implementations, numerous modifications, alterations, and changes to the described implementations are possible without departing from the sphere and scope of the present disclosure, as defined in the appended claim(s). Accordingly, it is intended that the present disclosure not be limited to the described implementations. Rather these implementations should be considered as illustrative and not restrictive in character. All changes and modifications that come within the spirit of the current subject matter are to be considered within the scope of the disclosure. The present disclosure should be given the full scope defined by the language of the following claims, and equivalents thereof. The discussion of any implementation is meant only to be explanatory and is not intended to suggest that the scope of the disclosure, including the claims, is limited to these implementations. In other words, while illustrative implementations of the disclosure have been described in detail herein, it is to be understood that the inventive concepts may be otherwise variously embodied and employed, and that the appended claims are intended to be construed to include such variations, except as limited by the prior art. Unless otherwise defined, all technical terms used herein have the same meaning as commonly understood by one of ordinary skill in the art to which the disclosure belongs.

Directional terms such as top, bottom, superior, inferior, medial, lateral, anterior, posterior, proximal, distal, upper, lower, upward, downward, left, right, longitudinal, front, back, above, below, vertical, horizontal, radial, axial, clockwise, and counterclockwise) and the like may have been used herein. Such directional references are only used for identification purposes to aid the reader's understanding of the present disclosure. For example, the term “distal” may refer to the end farthest away from the medical professional/operator when introducing a device into a patient, while the term “proximal” may refer to the end closest to the medical professional when introducing a device into a patient. Such directional references do not necessarily create limitations, particularly as to the position, orientation, or use of this disclosure. As such, directional references should not be limited to specific coordinate orientations, distances, or sizes, but are used to describe relative positions referencing particular implementations. Such terms are not generally limiting to the scope of the claims made herein. Any implementation or feature of any section, portion, or any other component shown or particularly described in relation to various implementations of similar sections, portions, or components herein may be interchangeably applied to any other similar implementation or feature shown or described herein.

It should be understood that, as described herein, an “implementation” (such as illustrated in the accompanying Figures) may refer to an illustrative representation of an environment or article or component in which a disclosed concept or feature may be provided or embodied, or to the representation of a manner in which just the concept or feature may be provided or embodied. However, such illustrated implementations are to be understood as examples (unless otherwise stated), and other manners of embodying the described concepts or features, such as may be understood by one of ordinary skill in the art upon learning the concepts or features from the present disclosure, are within the scope of the disclosure. Furthermore, references to “one implementation” of the present disclosure are not intended to be interpreted as excluding the existence of additional implementations that also incorporate the recited features.

In addition, it will be appreciated that while the Figures may show one or more implementations of concepts or features together in a single implementation of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one implementation can be used separately, or with another implementation to yield a still further implementation. Thus, it is intended that the present subject matter covers such modifications and variations as come within the scope of the appended claims and their equivalents.

As used herein, an element or step recited in the singular and proceeded with the word “a” or “an” should be understood as not excluding plural elements or steps, unless such exclusion is explicitly recited. It will be further understood that the terms “includes” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

The phrases “at least one,” “one or more,” and “and/or,” as used herein, are open-ended expressions that are both conjunctive and disjunctive in operation. The terms “a” (or “an”), “one or more” and “at least one” can be used interchangeably herein.

Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

The foregoing discussion has been presented for purposes of illustration and description and is not intended to limit the disclosure to the form or forms disclosed herein. For example, various features of the disclosure are grouped together in one or more implementations or configurations for the purpose of streamlining the disclosure. However, it should be understood that various features of the certain implementations or configurations of the disclosure may be combined in alternate implementations or configurations. Moreover, the following claims are hereby incorporated into this detailed description by this reference, with each claim standing on its own as a separate implementation of the present disclosure.

What is claimed:

1. An impactor, comprising:
an electromagnetic component including a stationary electromagnetic housing and a moving armature component, wherein the stationary electromagnetic housing includes a coil configured to receive an electric current resulting in generation of an electromagnetic field for triggering translation of the armature component within the stationary electromagnetic housing;
a striker body configured to be coupled to the armature component and an object, the striker body including a distal end;
a strike chamber having an interior and a distal strike wall disposed at a distal end of the strike chamber;
a spacer disposed at a proximal end of the strike chamber and opposite the distal strike wall;
wherein the electromagnetic field is configured to force the armature component and the striker body to translate in at least one direction, wherein translation of the armature component is configured to cause the distal end of the striker body to strike at least one of the distal strike wall and the spacer.

2. The impactor of claim 1, wherein the at least one direction of translation movement of the armature component and the striker body is directly and/or indirectly dependent on a direction of the electric current applied to the coil.

3. The impactor of claim 2, wherein a change in a direction of the electric current applied to the coil is configured to change the at least one direction of translation movement of the armature component and the striker component.

4. The impactor of claim 1, wherein the at least one direction includes at least one of: a forward impact direction, a reverse impact direction, a combination of forward and reverse impact directions, or any combination thereof.

5. The impactor of claim 1, wherein the object includes at least one of: a tool, an implant, or any combination thereof.

6. The impactor of claim 1, wherein the translation movement of the striker body is configured for positioning an implant in a bone and/or removal of the implant from the bone.

7. The impactor of claim 1, wherein the coil is configured to receive one or more current pulses to trigger translation of the armature component and the striker body.

8. The impactor of claim 7, wherein the one or more current pulses include a high current pulse and a low current pulse, wherein the low current pulse is smaller than the high current pulse.

9. The impactor of claim 8, wherein a duration of application of the high current pulse is longer than a duration of application of the low current pulse.

10. The impactor of claim 9, wherein application of the low current pulse is configured to prevent movement of the armature component and the striker body in a direction opposite to a direction of movement of the armature component and the striker body resulting from application of the high current pulse.

11. The impactor of claim 8, wherein the high current pulse and the low current pulse are applied intermittently.

12. The impactor of claim 1, wherein the spacer is configured to limit a translation distance of the distal end of the striker body within the interior of the strike chamber.

13. The impactor of claim 1, further comprising one or more sensors configured to detect position of the armature component within the stationary electromagnetic housing.

14. The impactor of claim 13, wherein the at least one direction of translation of the armature component is determined based on the position detected by the one or more sensors.

15. An impactor, comprising:
an electromagnetic component including a stationary electromagnetic housing and a moving armature component, wherein the stationary electromagnetic housing includes a coil configured to receive an electric current resulting in generation of an electromagnetic field for triggering translation of the armature component within the stationary electromagnetic housing;
a striker body configured to be coupled to the armature component and an object, the striker body including a distal end;
a strike chamber having an interior and a distal strike wall disposed at a distal end of the strike chamber, wherein the electromagnetic field is configured to force the armature component and the striker body to translate in at least one direction, wherein translation of the armature component is configured to cause the distal end of the striker body to strike the distal strike wall;
one or more sensors configured to determine position of the moving armature component; and
at least one processor configured to determine an amount and a direction of the electric current to apply to the coil to generate the electromagnetic field based on data associated with the determined position of the moving armature component and received from the one or more sensors.

16. The impactor of claim 15, wherein the direction includes at least one of: a forward impact direction, a reverse impact direction, a combination of forward and reverse impact directions, or any combination thereof.

17. The impactor of claim 15, wherein the amount and the direction of current is determined at at least one of: prior to the translation of the armature component for delivery of an impact by the impactor, during the translation of the armature component for delivery of the impact, during the translation of the armature component after delivery of the impact, or after the translation of the armature component.

18. The impactor of claim 15, wherein, in response to the at least one processor determining the amount and the direction of the electric current, the coil is configured to receive one or more current pulses to trigger translation of the armature component and the striker body, wherein the one or more current pulses include a high current pulse and a low current pulse, wherein the low current pulse is smaller than the high current pulse.

19. The impactor of claim 18, wherein a duration of application of the high current pulse is longer than a duration of application of the low current pulse, wherein application of the low current pulse is configured to prevent movement of the armature component and the striker body in a direction opposite to a direction of movement of the armature component and the striker body resulting from application of the high current pulse.

20. The impactor of claim 18, wherein the high current pulse and the low current pulse are applied intermittently.
